(19) United States
(12) Patent Application Publication
(10) Pub. No.: US 2014/0227748 A1
(43) Pub. Date: Aug. 14, 2014

(54) METHOD FOR THE EPA ENRICHMENT OF MICROALGAE OF THE MONODUS GENUS CULTIVATED IN MIXOTROPHIC MODE

(71) Applicants: Pierre Calleja, Bordeaux (FR); Khadidja Romari, Clermont-Ferrand (FR)

(72) Inventors: Pierre Calleja, Bordeaux (FR); Khadidja Romari, Clermont-Ferrand (FR)

(73) Assignee: FERMENTALG, Liboume (FR)

(21) Appl. No.: 14/124,829

(22) PCT Filed: Jun. 7, 2012
(86) PCT No.: PCT/FR2012/051281

§ 371 Date: Feb. 19, 2014

(30) Foreign Application Priority Data
Jun. 8, 2011 (FR) 1154980

(51) Int. Cl. C12P 7/64; C12R 1/89

(52) U.S. Cl. CPC C12P 7/6427; C12R 1/89
USPC 435/134; 435/257.1

(57) ABSTRACT

Novel strains of microalgae belonging to the Monodus genus allow optimum production of polyunsaturated fatty acids, notably EPA, in mixotrophic mode. A method for selecting and culturing such strains, using a discontinuous supply of light in the form of flashes is also described.

The invention relates to a novel strain of microalga belonging to the Monodus genus, particularly suitable for producing fatty acids in mixotrophic culture mode.

This novel strain of Monodus is useful for producing EPA (eicosapentaenoic acid) in mixotrophic mode, notably in the presence of a discontinuous supply of light in the form of flashes.

### PREAMBLE

It is known that microalgae are photosynthetic microorganisms with an autotrophic character, i.e. they have the capacity to grow autonomously by photosynthesis.

Microalgae develop both in marine aquatic media, and in fresh or brackish waters, as well as in various land habitats.

Most of the microalgae species encountered in fresh water or in the oceans are strictly autotrophic, i.e. they can only grow by photosynthesis. For the latter, the presence in their environment of carbon-containing substrates or organic matter is not favorable to them and even tends to inhibit their growth.

However, a certain number of microalgae species, of very varied families and origins, are found to be not strictly autotrophic. Thus, some of them, said to be heterotrophic are capable of developing in the total absence of light, by fermentation, i.e. by using the organic matter.

Other microalgae species, for which photosynthesis remains indispensable for their development, are capable of benefiting from photosynthesis and from the organic matter present in their environment. These intermediate species, said to be mixotrophic, can be cultured both in the presence of light and organic matter.

This particularity of so-called [symbol] mixotrophic [symbol] algae seems to be related to their metabolism which allows them to carry out photosynthesis and fermentation simultaneously. Both types of metabolism coexist with a positive overall effect on the growth of the algae [Yang C. et al. (2000) Biochemical Engineering Journal 6:87-102].

At present, the classification of algae is still widely based on morphological criteria and on the character of the photosynthetic pigments which their cells contain. Consequently, it is not very indicative of the autotrophic, heterotrophic or mixotrophic character of the species of algae, whereas the latter cover a very great diversity of species and of forms [Dubinsky et al. 2010, Hydrobiologia, 639:153-171].

Microalgae are currently the subject of many industrial projects since certain species are capable of accumulating or secreting large amounts of lipids, notably, polyunsaturated fatty acids.

Among these polyunsaturated fatty acids, certain highly unsaturated acids of the series of Omega-3s (PUFA-ω3), in particular eicosapentaenoic acid (EPA, C20:5 ω3) and docosahexaenoic acid (DHA, C22:6 ω3) have a recognized nutritional importance and have strong potential in terms of therapeutic applications [Horrocks L. A. et al. (2000) Health Benefits of DHA. Pharmacol. Res. 40: 211-225].

Fish oils from the fish industry are presently the main commercial source for this type of fatty acids. However, while these oils find new applications (food supplement in aquaculture, integration in margarines), marine halieutic resources become scarce because of intensive fishing activity.

New sources for EPA and DHA, therefore, have to be sought in order to meet, in the future, the increasing demand from the market for this type of polyunsaturated fatty acids.

In addition to their capability of synthesizing fatty acids de novo, microalgae provide several advantages compared with fish oils: they may be cultured in vitro under controlled conditions, which allows production of a biomass with a relatively constant biochemical composition, and on the other hand, unlike fish oils, they do not have any unpleasant smell and their lipids contain no, or only very little, cholesterol.

Finally, the lipids produced by microalgae have a simpler profile of fatty acids than that of fish oils, which limits the steps for separating the fatty acids of interest.

The taxonomic classification of eukaryotic algae contains 14 phyla. Large variations exist among the referenced species of the different classes making up these phyla as regards the microalgae content of polyunsaturated fatty acids. Moreover, the relative proportions of EPA and DHA in the lipid profiles vary according to the species and the culture conditions.

The main microalgae of interest, producing EPA and DHA are marine species. However, among the hundreds of thousands of species of marine microalgae, only a small number have a high content of both of these fatty acids and sufficient capacity so as to be cultured in vitro. The species of interest are mainly Bacillariophytes (or diatoms) from marine phytoplankton. They are generally characterized by an active production of EPA, but often by quite low contents of DHA.

Although they are rich in α-linolenic acid (C18:3 ω3), fresh water microalgae generally do not contain any EPA or DHA. The only freshwater microalgae capable of producing them to this day seem to belong to the family of Pleurochloridaceae, notably in species of the Monodus genus [Pencreac'h et al. (2004) Les microalgues marines: source alternative d'EPA et de DHA (Marine microalgae: alternative source of EPA and DHA), Lipides, 11(2):118-222].

Japanese patent applications JP9252764 and JP60087798 thus describe strains of Monodus subterraneus cultured in autotrophic mode which may accumulate an amount of EPA ranging up to 3.8% of their dry weight. These strains were cultured under laboratory conditions, i.e. in inorganic culture media, in flasks or in bioreactors with low volume capacity with a continuous light supply.

In the perspective of an industrial utilization, such culture mode proves to be unsuitable. Indeed, in order to be cost-effective, the production of biomass should be able to be carried out in closed, large photo-bioreactors. However, such a culture mode is difficult to achieve in autotrophic mode, because when the density of the cells increases in the culture medium, the cells have increasing difficulty in capturing light from the outside of the reactor. It is then necessary to actively mix the culture medium, which requires significant energy expense.

An alternative to autotrophic cultures would be to practice heterotrophic cultures, i.e. in the absence of light with provision of energy in the form of carbon-containing substrates, or else, mixotrophic cultures, i.e. with a supply of light of lesser intensity and in the presence of a supply of organic substrate.

However, the existing Monodus strains, as well as the methods used to culture them, are not very suitable for mixotrophic cultures, and even less for heterotrophic cultures, for this kind of microalgae.

Thus, after many experiments under unusual light conditions and the addition of different substrates, the applicant has managed to isolate microalga strains of the Monodus genus, which may be cultured in mixotrophic mode, allowing, under the conditions of the present invention, optimum production of polyunsaturated fatty acids, notably, EPA.

A strain (FCC 757) representative of novel Monodus strains, thus isolated and selected, was deposited at the CCAP (Culture Collection of Algae and Protozoa, Scottish Association for Marine Science, Dunstaffnage Marine Laboratory, Oban, Argyll PA37 1QA, Scotland, United Kingdom) according to the provisions of the Treaty of Budapest, on May 27, 2011 under the accession number CCAP 843/3.

The culture and selection method, more particularly, consisted of culturing microalgae under mixotrophic conditions, in the presence of discontinuous illumination, notably in the form of flashes.

The close alternation of illuminated phases and dark phases, generally perceived as stressful for microalgae, surprisingly made it possible to obtain a high production of polyunsaturated fatty acids from Monodus strains. This application of the strains according to the invention opens the perspective of industrial production of polyunsaturated fatty acids, in particular, EPA, in fermenters, benefiting from reduced light supply, and should therefore make possible energy savings compared with autotrophic culture modes.

The various aspects of the advantages of the invention are detailed hereafter.

### DETAILED DESCRIPTION

The present invention thus relates to a method for enriching microalgae of the Monodus genus in polyunsaturated fatty acids, more particularly, with EPA, characterized in that it comprises the culture of a microalga of the Monodus genus in mixotrophic mode.

The culture in mixotrophic mode of this microalga is preferentially carried out in the presence of at least 5 mM, preferably, at least 10 mM, more preferentially, at least 20 mM and still more preferentially, more than 50 mM of a carbon-containing substrate. One skilled in the art knows how to determine the maximum concentrations of the carbon-containing substrate to be used. Generally, the culture in mixotrophic mode of this microalga may be carried out in the presence of 10-200 mM, preferably, between 20 and 50 mM of carbon-containing substrate.

A continual substrate supply is ensured during the culture, in order to allow the cells to accumulate a significant concentration of lipids. Additional substrate is added to the culture medium during the culture method so as to maintain a constant concentration of carbon-containing substrate in the culture medium. One skilled in the art knows how to determine the concentrations of the carbon-containing substrate to be added to the culture in order to maintain a constant concentration of the carbon-containing substrate in the culture medium. Generally, the culture may be thus carried out with cumulated concentrations of carbon-containing substrate from 5 mM to 1 M, preferably, from 50 mM to 800 mM, more preferentially, from 70 mM to 600 mM, and even more preferentially, from 100 mM to 500 mM.

The carbon-containing substrate preferentially comprises, in pure form or as a mixture, glucose, cellulose derivatives, lactate, starch, lactose, saccharose, acetate and/or glycerol. A particularly preferred carbon-containing substrate is sodium acetate.

The carbon-containing substrate contained in the culture medium may consist in complex molecules or in a mixture of substrates. The products from the biotransformation of starch, for example, from maize, wheat or potato, notably, starch hydrolyzates, which consist of small sized molecules, for example, form carbon-containing substrates which may be used for mixotrophic culture of the microalgae according to the invention.

This method is more particularly intended for using novel strains of microalgae of the Monodus genus (Phylum: Xanthophyceae, Order: Mischococcales, Family: Pleurochloridaceae) [ITIS Catalogue of Life, 2010] selected for their mixotrophic character, notably for their capability of being cultured with a light supply greater than 10 μE, in a mineral medium, for example BG11 medium [Allen, M. M. & Stanier, R. Y. 1968. Growth and division of some unicellular blue-green algae. J. Gen. Microbiol. 51: 199-202], in which is added a carbon-containing substrate. Preferably, the carbon-containing substrate comprises glycerol in a concentration that is equivalent to or greater than 5 mM.

These novel stains of Monodus may be isolated and selected according to the selection and culture method according to the invention described hereafter.

A representative strain of the Monodus strains according to the invention is the FCC 757 strain isolated by the applicant and deposited at the CCAP on May 27, 2011 under the accession number CCAP 848/3. Such strains are capable of producing significant quantities of EPA when they are cultured in mixotrophic mode.

According to ongoing taxonomic analyses, the CCAP 848/3 strain belongs to the species Monodus subterraneus. Nevertheless, taking into account its filiation with other Monodus species, the invention relates to any microalga species of the Monodus genus having a mixotrophic character, as described in the present application.

To the applicant's knowledge, the isolated Monodus strains according to the invention are the first described as being able, under mixotrophic conditions, to produce significant quantities of EPA which may represent more than 10% of the total lipids contained in the microalgae.

Moreover, the obtained biomass is generally from 10 to 50%, more often from 20 to 40%, greater than that of the same culture carried out in autotrophic mode.

The invention thus relates to a method for culturing microalgae of the Monodus genus in mixotrophic mode, notably with a view to producing polyunsaturated fatty acids, such as EPA.

This culture method proves to be particularly advantageous when microalgae are cultured in the presence of a variable or discontinuous light supply, in other words when the light flux provided to the cultured algae is variable or discontinuous over time.

Contrary to common beliefs, it appeared that variable or discontinuous illumination of the cultures, in particular, when used in culture in mixotrophic mode, had a favorable impact on the development of the algae and made it possible to increase the productivity of the latter, notably as far as their lipid production is concerned.

Without being bound by theory, the inventor believes that a discontinuous or variable light supply to the microalgae has the effect of causing a [symbol] stress [symbol] favorable to the synthesis of lipids. This phenomenon may be partly explained by the fact that, in nature, microalgae tend to accumulate lipid reserves to withstand the constraints of their environment.

By discontinuous illumination, it is meant illumination punctuated with periods of darkness. The periods of darkness may be more than one quarter of the time, preferably, half or more of the time, during which the algae are cultured.

According to a preferred aspect of the invention, the illumination is discontinuous and, more preferentially, in the form of flashes, i.e. over periods of short durations. The successive illumination phases are then generally comprised between 5 seconds and 10 minutes, preferably, between 10 seconds and 2 minutes, more preferentially, between 20 seconds and 1 minute.

According to another embodiment, the illumination may be variable, which means that the illumination is not interrupted by phases of darkness, and the light intensity varies over time. This light variation may be periodic, cyclic or even random.

According to the invention, the illumination may vary continuously, i.e. the light intensity is not constant and permanently varies over time. (dμmol (photons)/dt≠0).

According to the invention, it is also possible to have a light supply combining continuous and discontinuous illumination phases.

The invention is, in particular, directed to a method for culturing microalgae of the Monodus genus, characterized in that said algae are cultured in darkness with a discontinuous or variable supply of light over time, the intensity of which, in micromoles of photons, varies by an amplitude equal to or greater than 10 μmol.m⁻².s⁻¹ at a rate of several times per hour, preferably equal to or greater than 50 μmol.m⁻².s⁻¹, more preferentially, equal to or greater than 100 μmol.m⁻².s⁻¹. The common point of these different discontinuous or variable illumination modes lies in the fact that, according to the invention, the intensity of the light brought to the algae in culture, expressed in micromoles of photons per second per square meter (μmol.m⁻².s⁻¹), varies at least once within the same hour. The amplitude of this variation of the light intensity is generally greater than 10 μmol.m⁻².s⁻¹, preferentially, greater than or equal to 20 μmol.m⁻².s⁻¹, more preferentially, greater than or equal to 50 μmol.m⁻².s⁻¹. In other words, every hour, preferably, several times within the hour, the light intensity attains a high value and a low value, the difference between these values being equal to or greater than that indicated above. Preferably, said light intensity successively attains the values 50 μmol.m⁻².s⁻¹ and 100 μmol.m⁻².s⁻¹ every hour, more preferentially, the values 0 and 50 μmol.m⁻².s⁻¹, still more preferentially, the values 0 and 100 μmol.m⁻².s⁻¹.

It is known that 1 μmol.m⁻².s⁻¹ corresponds to 1 μE.m⁻².s⁻¹ (Einstein), a unit often used in the literature.

The light supply in the cultures may be obtained by lamps distributed around the external wall of the fermenters. A clock triggers these lamps for defined illumination times. The fermenters are preferentially located in a temperature-controlled enclosure, shielded from daylight.

As the applicant could ascertain, the fact that the thus selected strains have good growth capabilities in mixotrophic mode, in the presence of a discontinuous light, predisposes said strains to higher production of polyunsaturated fatty acids, notably EPA.

The culture method according to the invention thus allows selection of Monodus strains with a mixotrophic character, similar to the strain isolated by the applicant and deposited at the CCAP and having a high yield of polyunsaturated fatty acids.

This method is characterized in that it comprises one or several of the following steps:
culturing various strains of the Monodus genus in darkness with a discontinuous or variable supply of light over time, the intensity of which in micromoles of photons, preferentially varies by an amplitude equal to or greater than 50 μmol.m⁻².s⁻¹ at a rate of at least once per hour;
maintaining said culture over several generations;
isolating the strain(s) for which the number of cells has most increased during said generations.

To screen the strains, various Monodus strains may be cultured in parallel on microplates, in the same enclosure, with a precise monitoring of the conditions and of the development of the various cultures. It is, thus, easy to determine the response of the various strains to discontinuous illumination and, if necessary, upon adding one or several carbon-containing substrates into the culture medium. The strains, which react favorably to the discontinuous illumination and to the carbon-containing substrates, generally provide a better yield for the production of lipids in terms of quality (polyunsaturated fatty acids more abundant in the lipid profile) and in terms of quantity (the lipids contain a higher proportion of EPA).

The microalgae may be selected in a fermenter from a diversified pool of microalgae, and from which one aims to select the variants advantaged by the selection mode according to the invention, combining discontinuous or variable light with mixotrophic culture conditions. In this case, the culture is carried out by maintaining the microalgae in cultures over many generations, and then isolation of the components which have become a majority in the culture medium, is performed at the end of the culture.

The culture method according to the invention is more particularly characterized in that culture of the strains is carried out over several generations, preferably in mixotrophic mode, and, in that the cells loaded with lipids are harvested.

The invention thus also relates to the production of the lipids, notably fatty acids, via the culture of microalgae of the Monodus genus with a mixotrophic character, preferably cultured or selected according to the methods mentioned earlier, and then, the recovery of the thus cultured microalgae to extract therefrom the lipid content, in particular, EPA.

The methods for selectively extracting EPA and DHA are known to one skilled in the art and are, for example, described by Bligh, E. G. et Dyer, W. J. [A rapid method of total lipid extraction and purification (1959) Can. J. Biochem. Physiol 37:911-917].

The invention also relates to microalgae of the Monodus genus, enriched in polyunsaturated fatty acids, which may be obtained according to the method of the invention, as described earlier. The total lipids of such microalgae generally comprise more than 30%, often more than 40% and sometimes even more than 50% of EPA by dry weight.

### EXAMPLE

### Culture of Monodus Strains in a Bioreactor

The cultures are grown in 2 L usable capacity fermenters (bioreactors) with dedicated automatic equipment with computerized supervision. The pH of the system is adjusted by adding base (1N sodium hydroxide solution) and/or acid (1N sulfuric acid solution). The culture temperature is set to 23° C. Stirring is achieved using 3 stirring rotors placed on the shaft according to the Rushton configuration (3-blade propellers with down-pumping). The stirring rate and the aeration flow rate are regulated to a minimum of 100 rpm and a maximum of 250 rpm with Qmin=0.5 vvm/Qmax=2 vvm respectively. The bioreactor is equipped with an external lighting system surrounding the transparent tank. The intensity and the light cycles are controlled by dedicated automatic equipment with computerized supervision.

The reactors are inoculated with a preculture prepared on a mixing table (140 rpm) in a controlled-temperature enclosure (22° C.) and illuminated continuously at 100 μE m-2 s-1. Precultures and cultures are prepared in bioreactors in BG11 medium supplemented with 10 mM of NaHCO₃. The organic carbon used for the mixotrophic culture in a bioreactor is sodium acetate at concentrations of between 20 mM and 50 mM. The carbon-containing organic substrate is added in the culture medium, in [symbol] fed-batch [symbol] mode.

### Monitoring of Cultures

The total biomass concentration is monitored by measuring the dry mass (filtration on a Whatman GFC filter, and then oven drying in vacuo, 65° C. and −0.8 bars, for a minimum of 24 h before weighing).

Regarding the quantification of the total number of lipids, 10⁷ cells/mL were extracted. Methods for extracting lipids are known to one skilled in the art and are, for example, described by Bligh, E. G. et Dyer, W. J. [A rapid method of total lipid extraction and purification (1959) Can. J. Biochem. Physiol 37:911-917].

### Flashing Light

The light supply in the bioreactor cultures was obtained with LED lamps distributed around the external wall of the fermenters. A clock triggers these LEDs for illumination times or pulses between 10 and 100 μE. The light intensity of the flash system used in mixotrophy is the same as the one used in autotrophy (control).

| Strain Monodus | | Mixotrophy with |
|---|---|---|
| Subterraneus | Mixotrophy | flashes |
| Biomass | +250% | +300% |
| (% relative to | | |
| autotrophy) | | |
| Total lipids | +20% | +35% |
| (% relative to | | |
| autotrophy) | | |
| EPA (% relative | +10% | +40% |
| to autotrophy) | | |

1. Method for enriching a microalga of the Monodus genus in EPA (eicosapentaenoic acid), characterized in that it comprises the culture of a microalga of the Monodus genus in mixotrophic mode.

2. Method according to claim 1, characterized in that the culture in mixotrophic mode of said microalga of the Monodus genus is carried out in the presence of a carbon-containing substrate comprising at least 5 mM, preferably, at least 10 mM, and more preferentially, at least 20 mM glucose, cellulose, starch, lactose, saccharose, acetate and/or glycerol.

3. Method according to claim 2, characterized in that said carbon-containing substrate, present in the culture medium, comprises at least 5 mM glycerol.

4. Method according to claim 2, characterized in that said carbon-containing substrate, present in the culture medium, comprises at least 5 mM sodium acetate, preferably, at least 20 mM sodium acetate.

5. Method according to claim 1, characterized in that it comprises a step for recovering the thus cultured microalgae.

6. Method according to claim 1, characterized in that it comprises the following steps:
culturing one or several strains of the Monodus genus in darkness with a discontinuous or variable supply of light over time, the intensity of which in micromoles of photons varies by an amplitude of more than 50 μmol.m⁻².s⁻¹, at a rate of at least once per hour;
maintaining said culture over several generations in the presence of a carbon-containing substrate in the culture medium;
harvesting the thus obtained Monodus cells.

7. Method according to claim 6, characterized in that the supply of light is in the form of flashes.

8. Method according to claim 7, characterized in that the flashing consists in successive illumination phases with a duration comprised between 5 seconds and 10 minutes, preferably, between 10 seconds and 2 minutes, more preferentially, between 20 seconds and 1 minute.

9. Method according to claim 1, characterized in that said microalga of the Monodus genus corresponds to the FCC 757 strain, deposited on May 27, 2011 at the CCAP (Culture Collection of Algae and Protozoa) under the accession number CCAP 848/3.

10. Method according to claim 1, characterized in that said thus cultured microalgae are recovered in order to extract EPA (eicosapentaenoic acid) from their lipid content.

11. Microalga of the Monodus genus, characterized in that its total lipids comprise more than 40%, preferably, more than 50% of EPA by dry weight.

12. Microalga characterized in that it consists in an isolated strain of the Monodus genus corresponding to the FCC 757 strain, deposited on May 27, 2011 at the CCAP (Culture Collection of Algae and Protozoa) under the accession number CCAP 848/3.
